(19) United States
(12) Patent Application Publication
(10) Pub. No.: US 2024/0047037 A1
(43) Pub. Date: Feb. 8, 2024

(54) SYSTEM AND METHOD FOR DETERMINING A MODIFICATION OR A CHANGE OF AN INITIAL MYOPIA CONTROL SOLUTION USED BY A MYOPIC SUBJECT

(71) Applicant: Essilor International, Charenton-le-Pont (FR)

(72) Inventors: Yin Zhen Janice TOH, Singapore (SG); Damien PAILLE, Singapore (SG); Gilles GARCIN, Charenton-le-Pont (FR)

(73) Assignee: Essilor International, Charenton-le-Pont (FR)

(21) Appl. No.: 18/257,917

(22) PCT Filed: Dec. 15, 2021
(86) PCT No.: PCT/EP2021/086026

§ 371 Date: Jun. 16, 2023

(30) Foreign Application Priority Data
Dec. 18, 2020 (EP) 20306615.4

(51) Int. Cl. G16H 20/40; A61B 3/00

(52) U.S. Cl. CPC G16H 20/40; A61B 3/0025

(57) ABSTRACT

A system for determining a modification or a change of an initial myopia control solution used by a myopic subject, including a device for determining a real initial value and real ulterior value of an eye state parameter representative of the myopia degree of the eye of the subject at an initial time and at an ulterior time, one or more memories having in memory statistical data relative to the statistical evolution of the eye state parameter with time while the initial myopia control solution is implemented, one or more processors programmed to determine an expected ulterior value of a magnitude linked to the eye state parameter at the ulterior time based on the statistical data relative to the statistical evolution of the eye state parameter with time while the initial myopia control solution is implemented.

### TECHNICAL FIELD OF THE INVENTION

The invention relates to a system and a method for determining a modification or a change of an initial myopia control solution used by a myopic subject.

### BACKGROUND INFORMATION AND PRIOR ART

Numerous documents describe devices and methods for controlling myopia evolution in a subject, in particular in children.

Myopia control solutions designate the devices, products or methods used to slow down the progression of myopia in a subject.

Myopia occurs when the eyeball is too long, relative to the focusing power of the cornea and lens of the eye. This causes light rays to focus at a point in front of the retina, rather than directly on its surface.

As a result, distant objects are seen blurred by a myopic subject.

Different types of devices and products are known for slowing down myopia progression, such as ophthalmic lenses, contact lenses or drugs.

Moreover, different uses of these devices and products are known for slowing down myopia progression. Different myopia control solutions associating one or more devices and/or products and their specific use are therefore known and implemented by eye care professionals to try to limit the progression of myopia in given subjects.

Each subject may react differently to the different myopia control solutions. The different myopia control solutions known may therefore have different efficacy for different subjects. Moreover, the efficacy of each myopia control solution may vary with time and other conditions of implementation.

Monitoring the efficacy of the myopia control solution implemented for a subject is therefore necessary to ensure that an efficient myopia control solution is proposed to the subject at all times.

The modification or change of the initial myopia control solution implemented by a subject is usually decided by an eye care professional based on his experience and knowledge. It is therefore performed in a non uniform manner.

### SUMMARY OF THE INVENTION

In this context, one object of the invention is to provide a system for determining a modification or a change of an initial myopia control solution used by a myopic subject in an objective manner. This is designed to help the eye care professional to take the decision of modifying or changing the initial myopia control method.

This is achieved according to the invention by providing a system for determining a modification or a change of an initial myopia control solution used by a myopic subject, comprising:
a device for determining a real initial value and real ulterior value of an eye state parameter representative of the myopia degree of the eye of the subject at an initial time and at an ulterior time;
one or more memories and one or more processors,
said one or more memories having in memory statistical data relative to the statistical evolution of said eye state parameter with time while said initial myopia control solution is implemented,
said one or more processors being programmed to:
i) determine an expected ulterior value of a magnitude linked to said eye state parameter at said ulterior time based on said statistical data relative to the statistical evolution of said eye state parameter with time while said initial myopia control solution is implemented, and a real ulterior value of said magnitude,
ii) compare said real ulterior value of said magnitude with said expected ulterior value of said magnitude,
iii) determine said modification or change of said initial myopia control solution for said subject based on the result of this comparison.

In a general manner, the system according to the invention enables to improve the global strategy for limiting the progression of myopia in a subject by determining in an objective manner if a modification or change of myopia control solution is needed.

The effects and/or efficacy of the initial myopia control solution currently implemented by the subject are taken into account. The initial myopia control solution applied to the subject being predetermined, the system is programmed to determine if this initial myopia control solution should be modified or changed in order to ensure that a myopia control solution with optimal efficacy is applied to the subject at all times.

In the following, the wording «modifying the initial myopia control solution» will be used in the case where the initial myopia control solution is modified, for example by modifying an implementation parameter of this initial myopia control solution such as sphere value of a lens, dosage of a drug, duration of wear of a device . . . , and the wording «changing the initial myopia control solution» will be used when the initial myopia control solution is replaced by a different myopia control solution. Therefore, when the wording “modifying or changing the initial solution” is used, it is meant that the initial myopia control solution is either kept with an implementation parameter modified or replaced by another myopia control solution.

Many different basic myopia control solutions are known and will be described in the following description. The myopia control solutions considered in the following may comprise one basic myopia control solution or several basic myopia control solutions combined, that is to say, implemented simultaneously. This is described in more details hereafter.

When the initial myopia control solution is only modified, the myopia control solution remains of the same type. It may be the same single basic myopia control solution, as described hereafter, or the same combination of two or more basic myopia control solutions. For example, the use of drugs such as atropine is a basic myopia control solution that may be combined, that is to say used simultaneously, with other basic myopia control solutions, such as correcting accommodative lag, providing retinal stimulation, peripheral hyperopic defocus or providing myopic defocus or corneal reshaping.

When the initial myopia control solution is modified, at least one implementation parameter of one of the basic myopia control solutions of said initial myopia control solution is modified. For example, the concentration or dosage of atropine is modified and/or the defocus provided is modified.

In a general manner, the implementation parameters of the myopia control solutions described here may comprise:
concentration, dosage, frequency of instillation in the eye of a drug,
value of an optical feature of an ophthalmic lens such as sphere, addition, cut-off level of wavelength filter, distribution of power in the periphery, location and/or power of micro-lenses, dimension of scattering components, duration of wear of the ophthalmic lens,
curvature profile, duration of wear of a lens for corneal reshaping . . . .

When the initial myopia control solution is changed, the type of myopia control solution is changed. The change may include adding one or several basic myopia control solutions to the initial myopia control solution already in use or removing one or several basic myopia control solutions from the initial myopia control solution already in use, to obtain a different myopia control solution. The change may also include replacing at least one of the basic myopia control solutions implemented in said initial myopia control solution by at least one other basic myopia control solution.

Several studies suggest that, while many different initial myopia control solutions will have an effect reducing myopia progression for a subject, their efficacy tends to decrease with time or may provide a “rebound” effect after a first period of myopia progression reduction. During such a «rebound» effect, the increase of the myopia is accelerated compared to its progression without myopia control solution. Modifying or changing the initial myopia control solution implemented is a way of ensuring that the subject is provided with a myopia control solution having satisfactory efficacy at all times.

Basic myopia control solution and myopia control solutions comprising a combination of these basic myopia control solutions will be described in detail later.

Other advantageous and non-limiting features of the system according to the invention may be the following:
said step iii) of determination of modification or change of said initial myopia control solution comprises determining an appropriate moment to perform said modification or change and/or determining a modified or changed myopia control solution;
said one or more processors are programmed to:
   determine a high threshold value of statistically usual values for said magnitude at said ulterior time,
   compare said real ulterior value of the magnitude with said high threshold value, and
   determine said modification or change of said initial myopia control solution taking into account the result of this comparison;
said real ulterior value of the magnitude is comprised between said expected ulterior value and said high threshold value, said one or more processors are programmed to:
   when said magnitude increases when the myopia degree of the eye increases, determine said modification or change of said initial myopia control solution as comprising a modification of at least one implementation parameter of said initial myopia control solution
   and
   when said magnitude decreases when the myopia degree of the eye decreases, determine said modification or change of said initial myopia control solution as comprising no modification nor change of said initial myopia control solution;
when said real ulterior value of the magnitude is higher than said high threshold value, said one or more processors are programmed to:
   when said magnitude increases when of the myopia degree of the eye increases, determine said modification or change of said initial myopia control solution as comprising a change from said initial myopia control solution to a different myopia control solution and
   when said magnitude decreases when the myopia degree of the eye increases, determine said modification or change of said initial myopia control solution as comprising a modification of at least an implementation parameter of said initial myopia control solution or no modification of said initial myopia control solution or a change to no myopia control solution;
said one or more processors are programmed to:
   determine a low threshold value of statistically usual values for said magnitude at said ulterior time,
   compare said real ulterior value of the magnitude with said low threshold value, and
   determine said modification or change of said initial myopia control solution taking into account the result of this comparison.
when said real ulterior value of the magnitude is comprised between said expected ulterior value and said low threshold value, said one or more processors are programmed to:
   when said magnitude increases when the myopia degree of the eye increases, determine said modification or change of said initial myopia control solution as comprising no modification nor change of said initial myopia control solution and
   when said magnitude decreases when the myopia degree of the eye increases, determine said modification or change of said initial myopia control solution as comprising a modification of at least one implementation parameter of said initial myopia control solution;
when said real ulterior value of the magnitude is lower than said low threshold value, said one or more processors are programmed to:
   when said magnitude increases when the myopia degree of the eye increases, determine said modification or change of said initial myopia control solution as comprising a modification of at least an implementation parameter of said initial myopia control solution or no modification of said initial myopia control solution or a change to no myopia control solution and
   when said magnitude decreases when the myopia degree of the eye increases, determine said modification or change of said initial myopia control solution as comprising a change from said initial myopia control solution to a different myopia control solution;
said one or more processors are programmed to:
   determine a minimum or maximum threshold value of statistically usual values for said magnitude at said ulterior time,
   compare said real ulterior value of the magnitude with said minimum or maximum threshold value, and
   determine said modification or change of said initial myopia control solution taking into account the result of this comparison;
said one or more processors are programmed to determine said modification or change of said initial myopia control solution as comprising a modification of at least an implementation parameter of said initial myopia control solution or no modification of said initial myopia control solution or a change to no myopia control solution:
   when said magnitude increases when the myopia degree of the eye increases and said real ulterior value of the magnitude is lower than said minimum threshold value, and
   when said magnitude decreases when the myopia degree of the eye increases and said real ulterior value of the magnitude is higher than said maximum threshold value;
said eye state parameter comprises an optical and/or physical feature of the eye, or a combination or a ratio of values of an optical and/or physical feature of the eye, such as spherical equivalent refraction and/or axial length of the eye or a combination or a ratio of these or a cumulative reduction in axial elongation;
said magnitude linked to said eye state parameter comprises said eye state parameter or a combination and/or a ratio of values of said eye state parameter or a magnitude calculated taking into account at least one value of said eye state parameter;
said myopia control solution comprises a device for implementing one of the following actions or several of the following actions simultaneously:
   correcting or reducing the accommodative lag during near vision activities for example using a lens having a positive sphere power;
   correcting peripheral hyperopic defocus or providing myopic defocus for example by using a lens having a positive sphere power in the region corresponding to peripheral vision;
   providing retinal stimulation for example using multiple light stimuli located in front of retina of the eye of the subject;
   providing a different contrast in peripheral vision of the subject by for example using a lens having a lower transmission in the region corresponding to peripheral vision;
   limiting the amount of red light entering the eye to reduce chromatism of the eye for example using a specific filter,
   providing light having a specific wavelength to the eye to inhibit eye lengthening,
   providing dynamically varying light stimuli,
   providing drugs to regulate retinal and scleral muscarinic receptors;
   reshaping the cornea of the eye to reduce refraction defects;
   providing myopic defocus in the retinal periphery and/or optical aberrations by flattening the shape of the cornea;
said one or more memories comprises a database where at least one individual feature of the subject is recorded and said one or more processors are programmed to take into account said at least one individual feature to determine said expected ulterior value of said magnitude at said ulterior time and/or take into account said at least one individual feature to determine said modification or change of said initial myopia control solution for said subject;
said one or more memories comprises a database where at least one individual feature of the subject is recorded and said one or more processors are programmed to determine said initial myopia control solution, said determination comprising:
   using a predetermined relationship model configured for providing the efficacy of each myopia control solution taking into account the individual feature of the subject,
   comparing the efficacies determined and
   determining said initial myopia control solution based on this comparison.
said individual feature of the subject comprises one or more of the following:
   personal historical or physical features of the subject;
   optometric features of the subject;
   biometric features of the subject;
   general health features of the subject;
   features relative to a visual behavior of the subject
   postural features of the subject.

The invention also relates to a method for determining a modification or a change of an initial myopia control solution used by a myopic subject, comprising:
determining a real initial value and real ulterior value of an eye state parameter representative of the myopia degree of the eye of the subject at an initial time and at an ulterior time;
providing statistical data relative to the statistical evolution of said eye state parameter with time while said initial myopia control solution is implemented,
determining an expected ulterior value of a magnitude linked to said eye state parameter at said ulterior time based on said statistical data relative to the statistical evolution of said eye state parameter with time while said initial myopia control solution is implemented, and a real ulterior value of said magnitude,
comparing said real ulterior value of said magnitude with said expected ulterior value of said magnitude,
determining said modification or change to said initial myopia control solution for said subject based on the result of this comparison.

### DETAILED DESCRIPTION OF EXAMPLE(S)

The following description with reference to the accompanying drawings will make it clear what the invention consists of and how it can be achieved. The invention is not limited to the embodiments illustrated in the drawings. Accordingly, it should be understood that where features mentioned in the claims are followed by reference signs, such signs are included solely for the purpose of enhancing the intelligibility of the claims and are in no way limiting on the scope of the claims.

In the accompanying drawings:

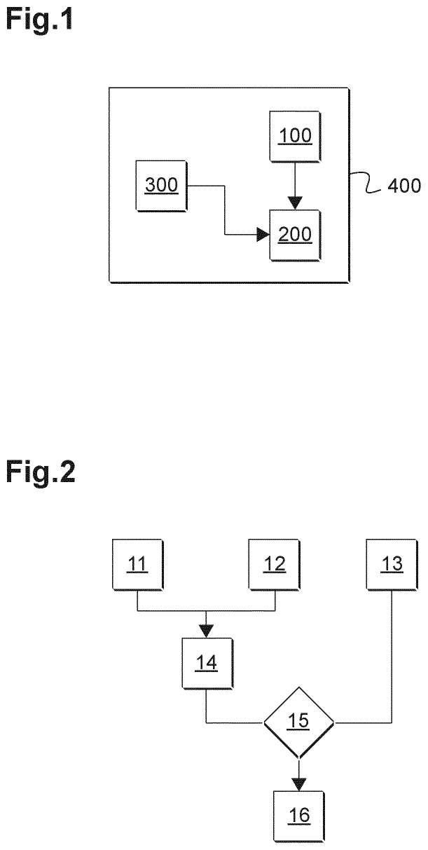

FIG. 1 is a schematic representation of the system according to the invention,

FIG. 2 is a schematic representation of the different steps of a method implemented by the system according to the invention,

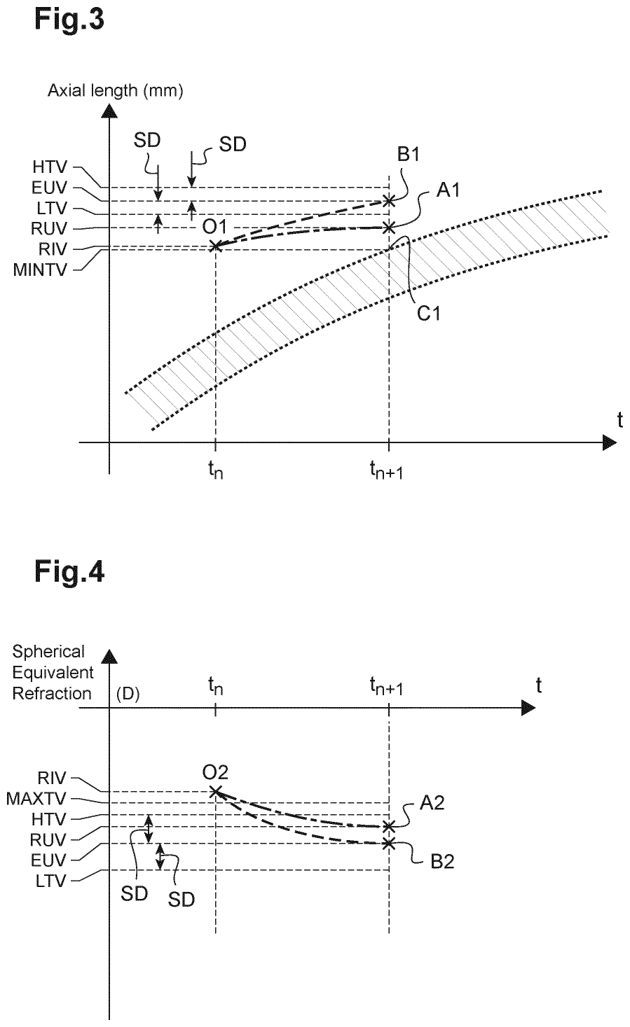

FIG. 3 is a schematic graphical representation of the comparison between the actual evolution with time of an eye state parameter representative of the myopia degree of the eye of the subject with said initial myopia control, here the axial length of the eye in mm (curve in long and short dashes), the expected evolution with time of said eye state parameter with said initial myopia control (curve in dashed lines), the mean normative zone of this eye state parameter for subjects with no myopia (hashed zone), and threshold values used in the method according to the invention,

FIG. 4 is a schematic graphical representation of the comparison between the actual evolution with time of an eye state parameter representative of the myopia degree of the eye of the subject with said initial myopia control, here the spherical equivalent refraction in diopters (curve in long and short dashes), the expected evolution with time of said eye state parameter with said initial myopia control (curve in dashed lines) and threshold values used in the method according to the invention.

FIG. 1 shows a schematic representation of the system 400 according to the invention.

As described hereafter, the system 400 according to the invention comprises:
a device 300 for determining a real initial value and real ulterior value of an eye state parameter representative of the myopia degree of the eye of the subject at an initial time and at an ulterior time;
one or more memories 100 and one or more processors 200,
said one or more memories 100 having in memory statistical data relative to the statistical evolution of said eye state parameter with time while said initial myopia control solution is implemented,
said one or more processors 200 being programmed to:
i) determine an expected ulterior value EUV of a magnitude linked to said eye state parameter at said ulterior time based on said statistical data relative to the statistical evolution of said eye state parameter with time while said initial myopia control solution is implemented, and a real ulterior value RUV of said magnitude,
ii) compare said real ulterior value RUV of said magnitude with said expected ulterior value EUV of said magnitude,
iii) determine said modification or change of said initial myopia control solution for said subject based on the result of this comparison.

The invention also relates to an associated method for determining a modification or a change of an initial myopia control solution used by a myopic subject, comprising:
determining a real initial value and real ulterior value of an eye state parameter representative of the myopia degree of the eye of the subject at an initial time and at an ulterior time;
providing statistical data relative to the statistical evolution of said eye state parameter with time while said initial myopia control solution is implemented,
determining an expected ulterior value EUV of a magnitude linked to said eye state parameter at said ulterior time based on said statistical data relative to the statistical evolution of said eye state parameter with time while said initial myopia control solution is implemented, and a real ulterior value RUV of said magnitude,
determining a real ulterior value of said magnitude and comparing it with said expected ulterior value of said magnitude,
determining said modification or change to said initial myopia control solution for said subject based on the result of this comparison.

The determination of modification or change of said initial myopia control solution may comprise:
determining an appropriate moment to perform said modification or change and/or
determining a modified or changed myopia control solution.

In particular, the determination of modification or change of said initial myopia control solution may comprise:
determining that a modification or a change is recommended at said ulterior time,
determining a modified or changed myopia control solution.

The initial myopia control solution is defined as the myopia control solution currently worn by the subject.

In an embodiment, said one or more processors are programmed to generate a signal indicating to the user that a modification or a change of myopia control solution is recommended. The signal then indicates that a modification or a change is recommended at once.

In another embodiment, said one or more processors are programmed to generate a signal indicating to the user that a modification or a change of myopia control solution is recommended at another time and the time at which it is recommended. The time is for example the next follow-up visit to an eye care professional.

The system and method according to the invention may then provide an indication of modification or change of said initial myopia control solution. In other words, the system indicates whether the initial myopia control solution currently implemented should be continued without modification or change or if it should be modified or changed.

Preferably, the system according to the invention indicates that the initial myopia solution should be either:
continued without modification,
continued with modification,
changed for another myopia control solution,
discontinued as well as any other myopia control solution.

In another embodiment, said one or more processors may be programmed to determine the modified or changed myopia control solution.

This determination of the modified or changed myopia control solution may be done for example according to the method described in WO2020/120595.

Here, the eye state parameter is representative of the myopia degree of the subject in the sense that it is linked to the myopia degree of the subject and varies with the myopia degree of the subject. The values of the eye state parameter may increase or, respectively, decrease with an increased myopia degree and decrease or, respectively, increase with a decreased myopia degree.

The myopia degree may be for example quantified by the absolute value of the refraction of the eye or lenses needed to correct the myopia of the eye. The degree of myopia may for example be described in terms of the power of the ideal dioptric correction needed by the eye, measured in diopters.

In practice, the eye state parameter representative of the myopia degree may comprise an optical and/or physical feature of the eye. It may in particular be based on the axial length of the eye, on the spherical equivalent refraction error of the eye of the subject, on the choroidal thickness of the eye or a combination or a ratio of these or cumulative reduction in axial elongation.

The axial length of the eye increases with an increased myopia degree and decreases with a decreased myopia degree, whereas the spherical equivalent refraction error decreases with an increased myopia degree and increases with a decreased myopia degree, as sphere power in myopia has a negative value.

The Cumulative Absolute Reduction in axial Elongation (CARE) is the cumulative treatment effect all along the treatment.

For example, if the axial elongation of the eye is 0.35 mm the first year, mm the second year and 0.25 mm the third year, the CARE after one year is mm the CARE after two years is 0.64 mm and the CARE after three years is mm.

The axial length of the eye (AL) is the distance between the anterior surface of the cornea and the fovea. It is usually measured by A-scan ultrasonography or optical coherence biometry.

The spherical equivalent refraction error is calculated by adding the sum of the sphere power with half of the cylinder power. For example, with a spectacle correction of −3.00−1.00×180, the spherical equivalent refraction error=−3.00 D+½ (−1.00 D)=−3.00 D−0.50 D=−3.50 D spherical equivalent refraction error.

Said device 300 for determining said real initial value and real ulterior value of said eye state parameter comprises preferably a device for measuring said real initial and ulterior value on said subject. In a variant, it may comprise a device programmed to retrieve these real initial and ulterior values or deduce them from measurements performed by another device, possibly in another location.

Said device 300 for determining said real initial value and real ulterior value of said eye state parameter comprises for example a phoropter and/or a biometric device or any device suitable for determining these values such as devices for A-scan ultrasonography or optical coherence biometry.

These devices and the methods for determining spherical equivalent refraction and/or axial length of the eye are well-known for the state of the art and not the object of the present invention. Therefore, they will not be described in details here.

Additionally, in an embodiment of said system 400 according to the invention:
said one or more memories comprises a database where at least one individual feature of the subject is recorded and
said one or more processors are programmed to take into account said at least one individual feature to determine said expected ulterior value of said magnitude at said ulterior time and/or take into account said at least one individual feature to determine said modification or change of said initial myopia control solution for said subject.

Said individual feature of the subject may comprise one or more of the following:
personal historical or physical features of the subject, such as number of myopic parent, age, gender, ethnicity, past or current optical equipment, pathologies, change in rate of myopia progression, change of the biological pattern of the eye, change in the On/Off receptive fields on the retina—optometric features of the subject, such as spherical or cylindrical refraction of the eye, prism, phoria, amplitude of accommodation, near point of convergence, dominant eye, peripheral refraction;
biometric features of the subject, such as eye axial length, size of the subject, interpupillary distance, cornea thickness;
ocular health features of the subject, such as dry eyes;
features relative to a visual behavior of the subject, such as time spent achieving near vision tasks or far vision tasks, activities practiced, usual time of day when using optical equipment, light environment, sleeping time
postural features of the subject such as viewing distance, reading distance between eyes and smartphones, books or notebooks, head or eye declination when performing some specific tasks such as reading, watching TV, working on a laptop.

In general, said initial myopia control solution is predetermined by any device or method known from the man skilled in the art.

Such a device/method for determining an initial myopia control solution is for example described in document WO2020/120595.

Alternatively, it may be determined by the system 400 according to the invention itself.

In a general manner, said one or more processors 200 are then programmed to determine the efficacy of each available myopia control solution, compare them and choose the one with the highest efficacy as the initial myopia control solution for said subject. Preferably, when said one or more memories 100 comprise a database where at least one individual feature of the subject is recorded, said one or more processors 200 are programmed to determine said initial myopia control solution using a predetermined relationship model configured for providing the efficacy of each myopia control solution taking into account the individual feature of the subject, comparing the efficacies determined and determining the initial myopia control solution as the one with the highest efficacy determined.

More precisely, said one or more memories 100 of said system according the invention may comprise a database where said individual features of the subject are recorded and said one or more processors is programmed to determine said initial myopia control solution, said determination comprising:
using a predetermined relationship model configured for providing the efficacy of each myopia control solution of said group of myopia control solutions taking into account the individual feature of the subject, and
comparing the efficacies of each myopia control solution of said group of myopia control solutions and
determining said initial myopia control solution based on this comparison.

Such a predetermined relationship model is for example described in WO2020/120595.

In a general manner, the predetermined relationship model may be updated regularly based on live/regular updates of new clinical trials and subjects test results, so as to dynamically adjust the expected efficacy of each myopia control solution based on these data.

Moreover, when said one or more memories comprise records of at least one individual feature of the subject in a database, said one or more processors may be additionally programmed to preselect, among said database of available myopia control solutions, a group of myopia control solution adapted to said subject based on said individual features, the efficacy of which is determined and compared.

The initial myopia control solution is then determined as the one with the highest efficacy among all the myopia control solutions of said group of myopia control solutions for said subject.

For example, the individual features of a child may indicate that he has a particularly thin cornea, that is to say, that the thickness of the cornea of this child is below a predetermined thickness threshold. Said one or more processors are then programmed to remove the myopia control solutions using a soft contact lens from the list of available myopia control solutions for said child. All other myopia control solutions are preselected.

As another example, for a child having a strong exophoria at near, that is to say, an exophoria above a predetermined exophoria threshold, said one or more processors are programmed to remove the bifocals and progressive addition lenses from the list of available myopia control solution for this child. All other myopia control solutions are preselected.

In the system 400 of the invention, said one or more memories 100 of the system 400 according to the invention comprise said statistical data relative to the statistical evolution of said eye state parameter with time while said initial myopia control solution is implemented.

In practice, said one or more memories 100 comprise a database of available myopia control solutions and statistical data relative to the statistical evolution of said eye state parameter with time while each of the available myopia control solutions is implemented.

Any known available myopia control solution may be taken into account in the system according to the invention.

Up to date, many different myopia control solutions have been described and tested and are currently used to limit the progression of myopia in subjects.

The following list is therefore provided as an example and is not exhaustive nor limitative.

Said myopia control solution may comprise a device for implementing one of the following actions or several of the following actions simultaneously. Each following actions correspond to a basic myopia control solution:
a) correcting or reducing the accommodative lag during near vision activities;
b) correcting peripheral hyperopic defocus or providing myopic defocus;
c) providing retinal stimulation with light using multiple light stimuli located in front of retina of the eye of the subject;
d) providing a different contrast in peripheral vision of the subject by using a lens having a lower transmission in the region corresponding to peripheral vision;
e) limiting the amount of red light entering the eye to reduce chromatism of the eye;
f) providing light having a specific wavelength to the eye to inhibit eye lengthening;
g) providing dynamically varying light stimuli such as flickering in order to decrease the contrast on the periphery of the retina by activating/deactivating diffusing elements according to the flickering frequency;
h) providing drugs to regulate retinal and scleral muscarinic receptors of the eye;
j) reshaping the cornea of the eye to reduce refraction defects;
k) providing myopic defocus in the retinal periphery and/or optical aberrations by flattening the shape of the cornea.

The accommodative lag of the eye is defined as the difference between accommodative demand and accommodative response and resulting in a residual refractive error.

Each myopia control solution may comprise one or several basic myopia control solutions as described above.

In practice, these actions are performed with five types of devices for controlling myopia:
Ophthalmic lenses integrated to spectacles,
Contact lenses worn directly on the eye of the subject,
Light sources for example to reproduce daily light indoors or avoid emitting wavelengths responsible for eye elongation,
Filters such as blue-cut filters cutting wavelengths responsible for eye elongation, for example integrated to a lens or comprising digital screen filters,
Pharmaceutical products.

Each device for controlling myopia may perform one or several actions listed above. Each myopia control solution may use one or several devices for controlling myopia.

For example, correcting or reducing the accommodative lag during near vision activities is done using a lens having a positive sphere power in the region of the lens used in near vision tasks. It may be a bifocal lens comprising a zone with a positive sphere located in the near vision region of the lens or a progressive lens with addition in the near vision region.

An example of bifocal lenses used to control myopia may be a prismatic bifocal lens having two optical zones: the zone located in the far vision region (upper part of the lens) compensate the myopic refraction defect of the eye, whereas the zone located in the near vision (lower part of the lens) region reduces the accommodation demand to see at near end and therefore the accommodative lag.

Correcting peripheral hyperopic defocus or providing myopic defocus may be achieved by using a lens having a positive sphere power in the periphery of the lens.

Customized lenses using multiple light stimuli located in front of retina of the eye of the subject may be used, which comprises:
a refraction area having a first refractive power based on a prescription for correcting an abnormal refraction of said eye; and
a plurality of at least three optical elements, at least one optical element having an optical function of not focusing an image on the retina of the eye so as to slow down the progression of the abnormal refraction of the eye.

Providing a different contrast in peripheral vision may be achieved by inserting scattering elements in periphery of the lens.

Limiting the amount of red light entering the eye may be achieved using a specific filter, for example integrated to a lens.

Providing light having a specific wavelength to the eye to inhibit eye lengthening may be achieved by luminotherapy involving exposing eyes to light having a wavelength that triggers the dopamine receptors regulating eye elongation.

Providing dynamically varying light stimuli may be achieved by exposing eyes to sinusoidal modulation of white light according to a temporal frequency.

Providing drugs to regulate retinal and scleral muscarinic receptors may be achieved for example by providing topical atropine directly delivered in the eye of the subject using eye drops. Other pharmacological approaches trialed for myopia control include topical timolol, a nonselective beta-adrenergic antagonist, and oral 7-methylxanthine (7-MX), an adenosine antagonist. The effect of atropine for myopia control has been shown is several studies, such as the study of Wu P.-C., Chuang M.-N., Choi J., Chen H., Wu G., Ohno-Matsui K., Jonas J. B., Cheung C. M. G., 2019, «Update in myopia and treatment strategy of atropine use in myopia control», in Eye 33, 3-13.

The effect of the drugs that may be provided to the eye typically lasts for at least a few hours. This is why when implemented with one or several other basic myopia control solutions the use of drugs is considered simultaneous.

Corneal reshaping, also known as orthokeratology, is performed by fitting a specially designed gas permeable contact lens on the eye of the subject. The contact lens is typically flattened in order to reshape the cornea of the eye to reduce the myopic refraction defect. The contact lens is typically worn at night, so that the shape of the eye is corrected when the contact lens is removed in the morning.

Providing myopic defocus in the retinal periphery and/or optical aberrations by flattening the shape of the cornea may be achieved for example by using orthokeratology.

Any other myopia control solution known for the man skilled in the art may be taken into account, and therefore included in said database in said memory 100.

In particular, all possible combinations of basic myopia control solutions may be taken into account, and therefore included in said database in said memory 100. Usually, combinations of two basic myopia control solutions are considered, such as the combination of the use of pharmaceutical products such as atropine in combination with any other myopia control solution. The combination of lenses providing retinal stimulation with the use of a positive sphere zone in the near vision region could also be considered.

Said statistical data may comprise a mean value of a magnitude linked to said eye state parameter for each myopia control solution, or the evolution with time of such a magnitude linked to said eye state parameter of each myopia control solution. It preferably also comprises data relative to the dispersion of the magnitude values, such as the evolution with time of the standard deviation of said magnitude while the initial myopia control solution is implemented.

Said statistical data may be retrieved during a clinical trial of each myopia control solution. Said clinical trial involves a control group of subjects and a test group of subjects.

The subjects of the control group are provided with a standard myopia correction method, whereas the subjects of the test group are provided with the myopia control solution whose efficacy is to be determined.

For example, subjects of the control group are children wearing classical ophthalmic single vision lenses, whereas the subjects of the test group are provided with a myopia control solution, such as lenses providing retinal stimulation using multiple light stimuli located in front of retina of the eye of the subject may be used.

The evolution of the myopia is followed for the subjects of each of the control group and test group by determining, at different times during the clinical trial, the value of a magnitude representative of the myopia degree of the eyes of each subject of the control group and test group.

Said magnitude linked to said eye state parameter may comprise said eye state parameter or a combination and/or a ratio of values of said eye state parameter or a magnitude calculated taking into account at least one value of said eye state parameter.

The axial length of the eye or the axial elongation (difference between current value of the axial length and a reference value) are examples of such magnitude representative of the myopia degree of the eyes. The spherical equivalent refraction or the absolute value of the spherical equivalent refraction of the eye is another example. Said magnitude representative of the myopia degree of the eyes may also, for example, be determined based on the power of the ideal dioptric correction needed by the eye, measured in diopters.

Therefore, said statistical data relative to the statistical evolution of said eye state parameter with time while said initial myopia control solution is implemented may for example comprise the mean evolution with time of said eye state parameter while the initial myopia control solution is implemented. Said statistical data may also comprise data relative to the dispersion of the eye state parameter values, such as the evolution with time of the standard deviation of said eye state parameter while the initial myopia control solution is implemented.

Said magnitude may also comprise an effective evolution parameter of said eye state parameter, as defined hereafter, or a magnitude quantifying the efficacy of the myopia control solution.

The efficacy of said myopia control solution is defined as a magnitude representative of the ability of this myopia control solution to produce a desired or intended result in controlling the myopia of the wearer. The efficacy may for example be quantified as a percentage value, 100% being the highest efficacy and 0% being the lowest efficacy.

Typically, the efficacy of a myopia control solution may be determined during said clinical trial involving a control group of subjects and a test group of subjects.

A mean current value of said magnitude representative of the myopia degree of the subjects may then be determined for each group, at each time t.

The efficacy of the myopia control solution tested at time t may then be determined based on the difference between the mean current values at time t of said magnitude representative of the myopia degree of the eye for the control and test groups. The efficacy may be equal to the absolute value of this difference or to the ratio between this difference and the mean current value of the magnitude representative of the myopia degree of the subjects of the control group. An effective evolution parameter is defined as the difference between two real values of the eye state parameter measured at different moments in time for the same wearer. The effective evolution parameter may be determined by comparing the ulterior value of the eye state parameter and to the initial value of this eye state parameter.

An absolute effective evolution parameter is equal to this difference, whereas a percentage effective evolution parameter is equal to the ratio between this difference and the initial value of the eye state parameter.

The absolute effective evolution parameter at an ulterior time is then equal to the real ulterior value of the eye state parameter minus the real initial value of this eye state parameter. The percentage effective evolution parameter at an ulterior time is equal to the absolute effective evolution parameter at this ulterior time divided by the real initial value of the eye state parameter.

These statistical data may be recorded under the form of pluralities of discrete values, under the form of curves representing graphically the statistical data or said standard deviation with time, or under the form of mathematical formulas, for example linking said eye state parameter, said magnitude or said standard deviation with time.

According to the method of the invention, said real initial value of said eye state parameter is provided (block 12 of FIG. 2), and said statistical data relative to the statistical evolution of said eye state parameter with time while said initial myopia control solution is implemented is provided (block 11 of FIG. 2).

On FIG. 3, said real initial value of the axial length of the eye is represented by point O1 at time tₙ. On FIG. 4, said real initial value of the spherical equivalent refraction error of the eye is represented by point O2 at time tₙ.

Said one or more processors 200 of the system according to the invention are programmed to determine, in step i) (block 14 of FIG. 2), an expected ulterior value of a magnitude linked to said eye state parameter at said ulterior time based on said statistical data relative to the statistical evolution of said eye state parameter with time while said initial myopia control solution is implemented.

Said expected ulterior value of the magnitude corresponds to the value of the magnitude that can be expected at said ulterior time when implementing the initial myopia control solution based on the statistical data. It is the value reached by said magnitude at said ulterior time when the initial myopia control solution has its statistical effect.

Said expected ulterior value for one particular myopia control solution may be calculated as equal to a real initial value of said magnitude, calculated taking into account the real initial value of the eye state parameter, or measured at said initial time, plus the mean evolution of the test group according to the clinical trial.

In the examples described here, said magnitude may be:
the axial length or spherical equivalent refraction or choroidal thickness of the eye,
an effective evolution parameter, for example the absolute effective evolution parameter or effective evolution parameter percentage as defined above, representative of the evolution of the myopia degree of the eye with time, for example determined based on axial length or spherical equivalent refraction or choroidal thickness,
a parameter linked to the efficacy of the myopia control solution.

In an embodiment, said magnitude increases when the myopia degree of the eye increases. Said magnitude may be for example the eye state parameter itself, such as the axial length. In step i), an expected ulterior value of the eye state parameter at said ulterior time is determined based on said real initial value of said eye state parameter and on said statistical data.

As an example, on FIG. 3, the statistical evolution of the axial length with time is represented by the curve linking point O1 and point B1. At time tₙ₊₁, tₙ₊₁ occurring after tₙ, the expected ulterior value EUV is represented by point B1. It is graphically determined or calculated based on the real initial value RIV by considering that the evolution of the eye state parameter with time corresponds to said statistical evolution.

For example, said statistical evolution may comprise the mean evolution of the eye state parameter. The expected ulterior value may be determined as the mean value of the eye state parameter at said ulterior time when said eye state parameter has the real initial value at said initial time.

In the example shown on FIG. 3, the statistical evolution of the eye state parameter with time when said initial myopia solution is implemented shows that the expected ulterior values of the eye state parameter are getting closer to the mean normative zone of this eye state parameter with time. This shows that the myopia evolution is statistically slowed down by the initial myopia control solution.

In the example described here, the eye state parameter is the axial elongation of the eye of the subject. An initial value of the axial elongation is measured at said initial time tₙ. If the mean evolution of the axial elongation indicates that after one year of using the initial myopia control solution, the axial elongation usually increases by 0.5 millimeter (mm), the expected ulterior value of the axial elongation of the eye of the subject is determined by said one or more processors to be the initial value plus 0.5 mm after one year, that is to say at tₙ₊₁=tₙ+one year.

In another embodiment, said magnitude decreases when the myopia degree of the eye increases. Said magnitude may for example be the eye state parameter itself, such as the spherical equivalent refraction error or the effective evolution parameter based on axial length.

As an example of this other embodiment, on FIG. 4, the statistical evolution of the spherical equivalent refraction error with time is represented by the curve linking point O2 and point B2. At time tₙ₊₁, tₙ₊₁ occurring after tₙ, the expected ulterior value EUV is represented by point B2. It is graphically determined or calculated based on the real initial value RIV by considering that the evolution of the eye state parameter with time corresponds to said statistical evolution.

In step i), an expected ulterior value of the magnitude at said ulterior time is determined based on the statistical data, that is to say calculated based on an expected ulterior value of the eye state parameter and the real initial value of this eye state parameter, or extracted from the statistical data.

In step ii), said one or more processors 200 are programmed to compare (block 15 of FIG. 2) a real ulterior value of said magnitude with said expected ulterior value of said magnitude.

In both embodiments, in the case where the magnitude is the eye state parameter itself, said real ulterior value of the eye state parameter is compared with the expected ulterior value of said eye state parameter.

In the case where the magnitude is the effective evolution parameter determined based on the eye state parameter, a real ulterior value of the effective evolution parameter at said ulterior time is determined based on the real initial and ulterior values of the eye state parameter and compared with the expected ulterior value of said effective evolution parameter.

In the example of FIG. 3, the real ulterior value RUV of the magnitude is represented by point A1. This real ulterior value is for example the value of the axial length of the eye of the subject measured after one year of using the initial myopia control solution. This real ulterior value is for example equal to the initial value plus 1 mm.

In the example of FIG. 4, the real ulterior value RUV of the magnitude is represented by point A2. This real ulterior value is for example the value of the spherical equivalent refraction error of the eye of the subject measured after one year of using the initial myopia control solution. This real ulterior value is for example equal to the real initial value RIV plus −0.25 D.

In this case the maximum threshold value may be equal to a real initial value of the magnitude.

Said comparison may be performed graphically or by a calculation, for example by calculating the difference between said expected ulterior value and said real ulterior value.

More precisely, said comparison may imply to:
determine a high threshold value HTV of statistically usual values for said magnitude at said ulterior time,
compare said real ulterior value RUV of said magnitude with said high threshold value HTV, and
determine said modification or change of said initial myopia control solution taking into account the result of this comparison.

Additionally, said one or more processor may be programmed to:
determine a low threshold value LTV of statistically usual values for said magnitude at said ulterior time,
compare said real ulterior value RUV of said magnitude with said low threshold value LTV, and
determine said modification or change of said initial myopia control solution taking into account the result of this comparison.

In practice, the comparison may indeed take into account statistical features of said statistical evolution with time of said magnitude when the initial myopia control solution is implemented, and the real ulterior value of said magnitude.

These statistical features allow determining said high and/or low thresholds corresponding to the range of statistically usual values for said magnitude at said ulterior time.

For example, high and/or low thresholds may take into account the standard deviation of said magnitude, eye state parameter or effective evolution parameter at said ulterior time. Said standard deviation may be a mean standard deviation or the value of the standard deviation at said ulterior time.

Said high threshold value HTV may correspond to the expected ulterior value EUV plus the standard deviation value SD at said ulterior time, i.e. HTV=EUV+SD, or plus a predetermined number multiplied by the standard deviation value at said ulterior time.

Said low threshold value LTV may correspond to the expected ulterior value EUV minus the standard deviation value at said ulterior time, i.e. HTV=EUV−SD, or minus a predetermined number multiplied by the standard deviation value at said ulterior time, said standard deviation value is defined as an absolute value in this application.

Said one or more processors 200 are then programmed to compare said real ulterior value RUV of said magnitude with the expected ulterior value EUV plus or minus the standard deviation value SD at said ulterior time, or plus or minus a predetermined number multiplied by the standard deviation value at said ulterior time.

For example, the real ulterior value RUV is compared to said expected ulterior value EUV and/or said expected ulterior value EUV plus one standard deviation SD, and/or said expected ulterior value plus or minus twice the standard deviation SD.

Optionally, said one or more processor may be programmed to:
determine a minimum MINTV or maximum MAXTV threshold value of statistically usual values for said magnitude at said ulterior time,
compare said real ulterior value of said magnitude with said minimum or maximum threshold value, and
determine said modification or change of said initial myopia control solution taking into account the result of this comparison.

Said minimum threshold value MINTV of statistically usual values for said magnitude at said ulterior time corresponds for example to a value linked to the mean normative value. For example, the one or more processors may be programmed to compare the real ulterior value RUV of the magnitude to a maximum normative value of the magnitude, that is to say to a maximum value statistically measured for subject having no myopia.

This maximum normative value corresponds to the mean normative value at said ulterior time plus a standard deviation value at this ulterior time. The maximum normative value at this ulterior time is represented by point Cl on FIG. 3 and corresponds to the minimum threshold MINTV.

In step iii), said one or more processors 200 are programmed to determine (block 16 of FIG. 2) said modification or change of said initial myopia control solution for said subject based on the result of this comparison.

According to an embodiment of the device of the invention, said one or more processors are programmed to:
when said real ulterior value RUV of the magnitude is higher than or equal to said high threshold value HTV, i.e. RUV≥HTV and said magnitude increases when the myopia degree of the eye increases, determine said modification or change of said initial myopia control solution as comprising a change from said initial myopia control solution to a different myopia control solution.

In the example described here, where the magnitude is the eye state parameter itself, such as the axial length, said high threshold value HTV is calculated to be the expected ulterior value EUV plus the corresponding standard deviation SD at said ulterior time, and said real ulterior value RUV of the eye state parameter is therefore compared to the expected ulterior value EUV plus the corresponding standard deviation SD at said ulterior time.

When said real ulterior value RUV of the eye state parameter is higher than the expected ulterior value EUV plus the corresponding standard deviation SD at said ulterior time, that is to say RUV>EUV+SD, said one or more processors are programmed to determine that said initial myopia control solution should be changed. In other words, the initial myopia control solution should be replaced by another myopia control solution.

This change may imply to change the device used to control said myopia for a different device. It may imply combining the initial myopia solution with another basic myopia solution. This change aims at providing a different myopia control solution having an increased effect on the subject.

For example, if said initial myopia solution consists in reshaping the cornea of the eye to reduce refraction defects through orthokeratology, it may be replaced by reshaping the cornea of the eye to reduce refraction defects through orthokeratology in combination with providing drugs to regulate retinal and scleral muscarinic receptors of the eye, for example with atropine drops.

Another example would be to determine a switch from contact lens solutions to spectacle solutions because of easiness to handle and maintain.

In an embodiment, said one or more processors are programmed to generate a signal indicating to the user that a change of myopia control solution is recommended.

In another embodiment, said one or more processors may be programmed to determine the changed myopia control solution. In order to determine the myopia control solution to which the initial myopia control solution should be changed, said one or more processors are programmed to take into account the expected efficacy of the available myopia control solutions.

In particular, as mentioned before said one or more memories 100 may comprise a database of available myopia control solutions. It may also comprise data relative to the efficacy of each myopia control solution.

In a general manner, said one or more processors 200 are programmed to determine the efficacy of each available myopia control solution, compare them and choose the one with the highest efficacy as the initial myopia control solution for said subject. The determination of the efficacy may take into account the individual features of the subject recorded in said one or more memories. Said one or more memories 100 of the system 400 according to the invention may also comprise data relative to the evolution of the efficacy of each myopia control solution with at least one criterion.

Said criterion may comprise at least one of the following: time spent using the initial myopia control solution and activity of the subject. The efficacy of each myopia control solution for a given value of said criterion may be for example estimated based on a predetermined relationship between the efficacy and said criterion.

This relationship might be a theoretical or empirical or measured relationship and is based on the previous clinical trial result. Said relationship then provides the evolution of the efficacy of each myopia control solution with said at least one criterion. Such a relationship is for example described in document WO2020/120595.

When said criterion is the time spent by the subject while using the initial myopia control solution, the evolution of the efficacy with said criterion corresponds to evolution of the efficacy with time, which is recorded in said memory 100.

It may be obtained thanks to a predetermined relationship linking the efficacy of each myopia control solution with the time spent using it, or through measurements allowing to determine the efficacy after different time spent using it.

When said criterion comprises the activity of the subject, said memory 100 comprises a table with the values of the efficacy of each myopia control solution for each activity of a list of possible activities. These values are preferably predetermined statistically. For example, it is the mean value of the efficacies determined through measurements for different subjects or through clinical trials.

The one or more processors are programmed to determine the changed myopia control solution as the available myopia control solution having the highest expected efficacy at said ulterior time, given the current value of said criterion.

In a simplified embodiment of the device according to the invention, with a magnitude increasing when the myopia degree of the eye increases, when said real ulterior value RUV of the magnitude is lower than the expected ulterior value EUV plus the corresponding standard deviation SD and higher than the expected ulterior value EUV at said ulterior time tₙ₊₁, said one or more processors are programmed to determine a modification of the initial myopia control solution to increase its effect on the subject.

In another simplified embodiment where the magnitude increases when the myopia degree increases, if the real ulterior value RUV of the magnitude is lower than the expected ulterior value of magnitude EUV and higher than the expected ulterior value EUV minus the corresponding standard deviation SD at said ulterior time tₙ₊₁, said one or more processors are programmed to determine no modification nor change of the initial myopia control solution.

In a preferred embodiment however, said magnitude increasing when the myopia degree of the eye increases, said one or more processor is programmed to:
when said real ulterior value RUV of the magnitude is comprised between said expected ulterior value EUV and said high threshold value HTV, i.e. EUV<RUV<HTV, determine said modification or change of said initial myopia control solution as comprising a modification of at least one implementation parameter of said initial myopia control solution.

In this case, the initial myopia control solution did not work as well as expected. The modification aims at making it more powerful (increased ADD, increased lenslet power, more lenslets, . . . )

More precisely, in the current example, when said real ulterior value of the eye state parameter, i.e. axial length, is lower than the expected ulterior value plus the corresponding standard deviation at said ulterior time and higher than the expected ulterior value, said one or more processors are programmed to determine a modification of said initial myopia control solution.

The system according to the invention may be programmed to provide an indication that a modification is needed, and, optionally, to determine said modified myopia control solution.

In an embodiment, said one or more processors are programmed to generate a signal indicating to the user that a modification of said initial myopia control solution is recommended.

In another embodiment, said one or more processors may be programmed to determine the modified myopia control solution.

Said modification of the initial myopia control solution implies no change in the nature of the myopia control solution already implemented, but rather a modification of the implementation parameters of the initial myopia control solution. This modification aims at increasing the effect of the initial myopia control solution or its efficacy. It may also aim at increasing compliance of the subject to the myopia control solution. The modification may lead to a decrease in visual quality.

For example, the expected value of the eye state parameter may have not been reached because of a lack of compliance to the recommended usage of the initial myopia control solution provided by the practitioner or the solution manufacturer. In the case of spectacle lenses, it can be that eyeglasses are not worn long enough during the day or not every day. One parameter to be modified can be the frame in order to have a more stable or comfortable one.

Other possible modifications may include:
increased addition power or more positive sphere power for solutions relating to correcting or reducing accommodative lag during near vision activities;
increased positive sphere power for solutions correcting peripheral hyperopic defocus in the region corresponding to peripheral vision;
increased number of lenslets and/or the positive range of power of the lenslets for solutions providing retinal stimulation using multiple light stimuli located in front of retina of the eye of the subject;
lowered transmittance in the periphery of the lenses to increase contrast for solutions providing a different and lower contrast in peripheral vision of the subject;
decreased amount of red light for solutions limiting the amount of red light entering the eye to reduce chromatism of the eye using a specific filter;
increased transmission of the wavelength for solutions providing light having a specific wavelength to the eye to inhibit eye lengthening;
increased frequency for solutions providing dynamically varying light stimuli such as flickering;
increased dosage for solutions providing drugs to regulate retinal and scleral muscarinic receptors;
modification of the shape of the lenses used in orthokeratology solutions to provide a flatter base curve and/or a, flatter center zone.

The system may indicate the above described appropriate modification to the user, depending on the initial myopia control solution.

In the current example, said one or more processor is moreover programmed to:
when said magnitude increases when the myopia degree of the eye increases and said real ulterior value RUV of the magnitude is comprised between said expected ulterior value EUV and said low threshold value LTV, determine said modification or change of said initial myopia control solution as comprising no modification nor change of said initial myopia control solution.

More precisely, in the current example, when said real ulterior value RUV of the eye state parameter, i.e. axial length, is comprised between said expected ulterior value EUV and the expected ulterior value EUV minus the corresponding standard deviation SD at said ulterior time, that is to say when EUV−SD<RUV<EUV, said one or more processors are programmed to determine no modification nor change of said initial myopia control solution.

Indeed, if the real ulterior value of the eye state parameter is within the range between said expected ulterior value and said low threshold value, the effect of the initial myopia control solution is satisfactory so far, and the initial myopia control solution should be continued without modification.

Preferably, said one or more processor is moreover programmed to:
when said magnitude increases when the myopia degree of the eye increases, and said real ulterior value RUV of the magnitude is lower than said low threshold value LTV, determine said modification or change of said initial myopia control solution as comprising a modification of at least an implementation parameter of said initial myopia control solution or no modification of said initial myopia control solution or a change to no myopia control solution (stop the initial myopia control solution).

Said modification is in this case designed to decrease the effect of the initial myopia control solution to increase the comfort of the wearer. This is especially the case when said real ulterior value A of the magnitude is comprised between said expected ulterior value EUV minus the corresponding standard deviation SD and the expected ulterior value EUV minus twice the corresponding standard deviation SD at said ulterior time, that is to say when EUV−2SD<RUV<EUV−SD.

Said modified or changed myopia control solution is then determined to provide a better visual quality, even if said modification of at least an implementation parameter of said initial myopia control solution decreases the effect of said initial myopia control solution or if said different myopia control solution has less effect.

The system according to the invention may be program to provide an indication that a modification or a change is needed, and, optionally, to determine said modified or changed myopia control solution.

To this end, possible modifications of the initial myopia solution may include:
decreased addition power or more positive sphere power for solutions relating to correcting accommodative lag during near vision activities;
decreased addition power or more positive sphere power for solutions relating to reducing accommodative lag during near vision activities;
decreased positive sphere power for solutions correcting peripheral hyperopic defocus in the region corresponding to peripheral vision;
decreased number of lenslets and/or the power of the lenslets for solutions providing retinal stimulation using multiple light stimuli located in front of retina of the eye of the subject;
increased transmittance in the periphery of the lenses to decrease contrast for solutions providing a different and lower contrast in peripheral vision of the subject;
increased amount of red light for solutions limiting the amount of red light entering the eye to reduce chromatism of the eye using a specific filter;
decreased transmission of the wavelength for solutions providing light having a specific wavelength to the eye to inhibit eye lengthening;
decreased frequency for solutions providing dynamically varying light stimuli such as flickering;
decreased dosage for solutions providing drugs to regulate retinal and scleral muscarinic receptors.

The system may indicate the above described appropriate modification to the user, depending on the initial myopia control solution.

Optionally, said one or more processor may also be programmed to:
when said magnitude increases when the myopia degree of the eye increases and said real ulterior value RUV of the magnitude is lower than said minimum threshold value MINTV, determine said modification or change of said initial myopia control solution as comprising a modification of at least an implementation parameter of said initial myopia control solution or no modification of said initial myopia control solution or a change to no myopia control solution.

In practice, in the current example described, when said real ulterior value of the eye state parameter such as axial length is lower than said maximum normative value, its means that the current value of the eye state parameter is within the range of statistically usual values of this eye state parameter for non myopic subjects. This means that the eye state parameter is within a “normal” range or myopia stopped progressing. Therefore, the system according to the invention is programmed to indicate that determining said modification or change of said initial myopia control solution as comprising a modification of at least an implementation parameter of said initial myopia control solution or no modification of said initial myopia control solution or a change to no myopia control solution. This modification of at least an implementation parameter aims at decreasing the effect of the initial myopia control solution so as to increase the comfort of the wearer.

Alternatively, said minimum threshold value could be set equal to the expected ulterior value EUV minus twice the corresponding standard deviation SD at said ulterior time or to the expected ulterior value EUV minus the corresponding standard deviation SD multiplied by a factor higher than two.

Preferably, said one or more processors are programmed to take into account the historical data of the subject. When said real ulterior value of the magnitude is determined to be lower than said minimum threshold value for the first time, said one or more processor is programmed to determine said modification or change of said initial myopia control solution as comprising a modification of at least an implementation parameter of said initial myopia control solution or no modification of said initial myopia control solution or a change to no myopia control solution as if said real ulterior value of the magnitude was lower than said low threshold value, in particular as if said real ulterior value RUV of the magnitude was comprised between said expected ulterior value EUV minus the corresponding standard deviation SD and the expected ulterior value EUV minus twice the corresponding standard deviation SD at said ulterior time.

Said one or more processors are programmed to determine a change to no myopia control solution when at least two real ulterior values of the eye state parameter are determined to be lower than said minimum threshold value with a two year gap between the determinations. If a real ulterior value is determined every 6 months, four determinations are done before stopping the myopia control. If a real ulterior value is determined every 12 months, two determinations are done before stopping the myopia control.

As mentioned before, the method according to the invention may comprises determining the modified or changed myopia control solution, and optionally, a final step of implementing the modified or changed myopia control solution.

The preselection of a group of myopia control solution adapted to the subject based on the individual features of the subject recorded may be applied at each appropriate step of the method described. The one or more processors are programmed accordingly.

The embodiments described here in association with the case where the magnitude is the eye state parameter itself may be applied to other magnitude as long as these other magnitudes vary with the myopia degree of the eye, that is to say increase or decrease in correspondence with a increased or decreased myopia degree.

In the case where said magnitude decreases when the myopia degree increases, the modifications or changes applied mirror the ones described above. As an example, the spherical equivalent and the choroidal thickness decreases as the myopia degree increases. The choroid, also known as the posterior portion of the uveal tract, is a vascular tissue that provides oxygen and nourishment to the outer portion of the retina and photoreceptors. The Choroidal thickness can be measured in vivo using ultrasonography, magnetic resonance imaging (MRI), and enhanced depth imaging optical coherence tomography (EDI-OCT)

Such a case is shown on FIG. 4. In this case, when said real ulterior value RUV of the magnitude is comprised between said expected ulterior value EUV and said high threshold value HTV, said one or more processors are programmed to determine said modification or change of said initial myopia control solution as comprising no modification nor change of said initial myopia control solution.

When said real ulterior value RUV of the magnitude is higher than said high threshold value HTV, said one or more processors are programmed to determine said modification or change of said initial myopia control solution as comprising a modification of at least an implementation parameter of said initial myopia control solution or no modification of said initial myopia control solution or a change to no myopia control solution. This modification aims at decreasing the effect of the initial myopia control solution so as to increase the comfort of the wearer. The change aims at implementing a different myopia control solution having a lower effect of the subject, but other advantages.

In practice, as described before, one can change one setting of the current solution (less powerful such as ADD, . . . ) or for an another type of solution with better visual quality.

When said real ulterior value RUV of the magnitude is comprised between said expected ulterior value EUV and said low threshold value LTV, said one or more processors are programmed to determine said modification or change of said initial myopia control solution as comprising a modification of at least one implementation parameter of said initial myopia control solution. This modification aims at increasing the effect of the initial myopia control solution. The change aims at implementing a different myopia control solution having a higher efficacy. In practice, as described before, the initial myopia control solution is modified to be more powerful (increase ADD, lenslet power/distribution, . . . )

When said real ulterior value RUV of the magnitude is lower than said low threshold value LTV, said one or more processors are programmed to determine said modification or change of said initial myopia control solution as comprising a change from said initial myopia control solution to a different myopia control solution. The change aims at implementing a different myopia control solution having a higher effect on the subject.

Said one or more processors are programmed to determine a modification of at least an implementation parameter of the initial myopia control solution so as to decrease its effect on the subject or no modification of the initial myopia control solution or a change to no myopia control solution when said real ulterior value of the magnitude is higher than said maximum threshold value.

Different ways of implementing the modification or changes in the initial myopia control solution have been described earlier and may be used for the embodiment where said magnitude decreases when the myopia degree of the eye increases as well.

As mentioned before, in all embodiments described here said step iii) of determination of modification or change of said initial myopia control solution may comprise determining an appropriate moment to perform said modification or change and/or determining a modified or changed myopia control solution.

In the case where said magnitude comprises an effective evolution parameter of an eye state parameter of the subject, for example based on the axial length of the eye or on the spherical equivalent refraction error of the eye of the subject, the appropriate time for modifying or changing the initial myopia control solution is for example determined during a follow-up visit scheduled by the prescriber with the subject upon delivery of said initial myopia control solution.

Parameters to be taken into account for defining the timeline for said follow-up visit can be according to:
the combination of age and myopia level: the higher the level of myopia is at a younger age implies a shorter period, for example equal to 3 months or less between two follow-up visits;
the myopia progression pace: the higher the risk of having a fast progression requires a shorter period before the next visit, for example equal to 3 months or less,
the expected efficacy of the myopia control solution: the lower the efficacy, the sooner is the follow-up visit to be determined for example equal to 3 months or less,
a predetermined timetable according to a standard myopia management program or package: regular visits to be done according to frequency that can be 3 months, 6 months or one year.

During each follow-up visit, the eye care practitioner preferably updates the individual features of the subject memorized in said system, in particular:
optometric parameters of the subject,
biometric parameters of the subject,
general health parameters of the subject,
visual behavior of the subject,
postural features of the subject

Updates can come from new measurements of monitoring devices used previously (daily wearing time, cumulative wearing time, postural data, . . . ).

An expected ulterior value of said effective evolution parameter at said ulterior time based on said statistical data and a real ulterior value of the effective evolution parameter may then be determined and compared.

Said time for modification or change and or said modification or change of said initial myopia control solution for said subject is determined based on the result of this comparison.

A detailed example based on usage of prismatic bifocal lenses using absolute values of effective evolution parameters on Spherical Equivalent Refraction could be the following.

At the first visit for a myopic child age of 8 years, prescription is the following:
Right Eye (RE): Sphere is −1.75 D, Cylinder is −0.50 D at 85°.
Left Eye (LE): Sphere is −2.50, Cylinder is OD.

The one or more processors are programmed to calculate the Spherical Equivalent Refraction of the right and left eyes at the initial time SER_RE(tₙ) and SER_LE(tₙ): SER_RE(tₙ)=−2.00 D and SER_LE(tₙ)=−2.50 D.

At the follow-up visit one year later:
Right Eye (RE): Sphere is −2.25, Cylinder is −0.75 D at 90°.
Left Eye (LE): Sphere is −2.75, Cylinder is OD.

The one or more processors are programmed to calculate the Spherical Equivalent Refraction of the right and left eyes at the ulterior time SER_RE(tₙ₊₁) and SER_LE(tₙ₊₁): SER_RE(tₙ₊₁)=−2.625 D and SER_LE(tₙ₊₁)=−2.75 D.

The corresponding absolute effective evolution parameters ABS_SER for each eye is equal to the difference between the Spherical Equivalent Refraction at ulterior and initial times: ABS_SER_RE=0.625 D and ABS_SER_LE=0.25 D.

According the statistical data recorded in said memories 100, for the Prismatic Bifocal Lens group, the average absolute effective evolution parameters is ABS_AVE=0.43 D and the dispersion is ASB_SD=0.03 D for one standard deviation after one year of using the prismatic bifocal lenses. The average absolute effective evolution parameters is therefore usually comprised between 0.4 D and 0.46 D for one standard deviation, between 0.37 D and 0.49 D for two standard deviations.

Therefore, for 95% of the tested population, after one year wearing a bifocal prismatic lens, SER is within 0.37 D and 0.49 D.

In this case, the initial myopia control solution did not work as expected for the right eye, as the value of the absolute efficacy ABS_SER_RE is above the high threshold of 0.49 D whereas it has been the case for the left eye as the value of the absolute effective evolution parameters ABS_SER_LE is below the low threshold of 0.37 D. The system is then programmed to indicate that a change of myopia control solution should be implemented in order to optimize the myopia control for both eyes.

Individual features such as gender, age may be taken into account to assess the expected efficacy.

1. A system for determining a modification or a change of an initial myopia control solution used by a myopic subject, comprising:
a device for determining a real initial value and real ulterior value of an eye state parameter representative of the myopia degree of the eye of the subject at an initial time and at an ulterior time;
one or more memories and one or more processors,
said one or more memories having in memory statistical data relative to the statistical evolution of said eye state parameter with time while said initial myopia control solution is implemented,
said one or more processors being programmed to:
i) determine an expected ulterior value of a magnitude linked to said eye state parameter at said ulterior time based on said statistical data relative to the statistical evolution of said eye state parameter with time while said initial myopia control solution is implemented, and a real ulterior value of said magnitude,
ii) compare said real ulterior value of said magnitude with said expected ulterior value of said magnitude, and
iii) determine said modification or change of said initial myopia control solution for said subject based on the result of this comparison.

2. The system according to claim 1, wherein said one or more processors are programmed to:
determine a high threshold value of statistically usual values for said magnitude at said ulterior time,
compare said real ulterior value of the magnitude with said high threshold value, and
determine said modification or change of said initial myopia control solution taking into account the result of this comparison.

3. The system according to claim 2, wherein, when said real ulterior value of the magnitude is comprised between said expected ulterior value and said high threshold value, said one or more processors are programmed to:
when said magnitude increases when the myopia degree of the eye increases, determine said modification or change of said initial myopia control solution as comprising a modification of at least one implementation parameter of said initial myopia control solution, and
when said magnitude decreases when the myopia degree of the eye increases, determine said modification or change of said initial myopia control solution as comprising no modification nor change of said initial myopia control solution.

4. The system according to claim 2, wherein, when said real ulterior value of the magnitude is higher than said high threshold value, said one or more processors are programmed to:
when said magnitude increases when the myopia degree of the eye increases, determine said modification or change of said initial myopia control solution as comprising a change from said initial myopia control solution to a different myopia control solution, and
when said magnitude decreases when the myopia degree of the eye increases, determine said modification or change of said initial myopia control solution as comprising a modification of at least an implementation parameter of said initial myopia control solution or no modification of said initial myopia control solution or a change to no myopia control solution.

5. The system according to claim 1, wherein said one or more processors are programmed to:
determine a low threshold value of statistically usual values for said magnitude at said ulterior time,
compare said real ulterior value of the magnitude with said low threshold value, and
determine said modification or change of said initial myopia control solution taking into account the result of this comparison.

6. The system according to claim 5, wherein when said real ulterior value of the magnitude is comprised between said expected ulterior value and said low threshold value, said one or more processors are programmed to:
when said magnitude increases when the myopia degree of the eye increases, determine said modification or change of said initial myopia control solution as comprising no modification nor change of said initial myopia control solution, and
when said magnitude decreases when the myopia degree of the eye increases, determine said modification or change of said initial myopia control solution as comprising a modification of at least one implementation parameter of said initial myopia control solution.

7. The system according to claim 5, wherein, when said real ulterior value of the magnitude is lower than said low threshold value, said one or more processors are programmed to:
when said magnitude increases when the myopia degree of the eye increases, determine said modification or change of said initial myopia control solution as comprising a modification of at least an implementation parameter of said initial myopia control solution or no modification of said initial myopia control solution or a change to no myopia control solution, and
when said magnitude decreases when the myopia degree of the eye increases, determine said modification or change of said initial myopia control solution as comprising a change from said initial myopia control solution to a different myopia control solution.

8. The system according to claim 1, wherein said one or more processors are programmed to:
determine a minimum or maximum threshold value of statistically usual values for said magnitude at said ulterior time,
compare said real ulterior value of the magnitude with said minimum or maximum threshold value, and
determine said modification or change of said initial myopia control solution taking into account the result of this comparison.

9. The system according to claim 8, wherein said one or more processors are programmed to determine said modification or change of said initial myopia control solution as comprising a modification of at least an implementation parameter of said initial myopia control solution or no modification of said initial myopia control solution or a change to no myopia control solution:
when said magnitude increases when the myopia degree of the eye increases and said real ulterior value of the magnitude is lower than said minimum threshold value, and/or
when said magnitude decreases when the myopia degree of the eye increases and said real ulterior value of the magnitude is higher than said maximum threshold value.

10. The system according to claim 1, wherein said eye state parameter comprises an optical and/or physical feature of the eye or a combination or a ratio of values of an optical and/or physical feature of the eye.

11. The system according to claim 1, wherein said magnitude linked to said eye state parameter comprises said eye state parameter or a combination and/or a ratio of values of said eye state parameter or a magnitude calculated taking into account at least one value of said eye state parameter.

12. The system according to claim 1, wherein said myopia control solution comprises a device for implementing one of the following actions or several of the following actions simultaneously:
correcting or reducing the accommodative lag during near vision activities;
correcting peripheral hyperopic defocus or providing myopic defocus;
providing retinal stimulation;
providing a different contrast in peripheral vision of the subject;
limiting the amount of red light entering the eye to reduce chromatism of the eye, providing light having a specific wavelength to the eye to inhibit eye lengthening,
providing dynamically varying light stimuli,
providing drugs to regulate retinal and scleral muscarinic receptors;
reshaping the cornea of the eye to reduce refraction defects;
providing myopic defocus in the retinal periphery and/or optical aberrations by flattening the shape of the cornea.

13. The system according to claim 1, wherein
said one or more memories comprise a database where at least one individual feature of the subject is recorded, and
said one or more processors are programmed to take into account said at least one individual feature to determine said expected ulterior value of said magnitude at said ulterior time and/or take into account said at least one individual feature to determine said modification or change of said initial myopia control solution for said subject.

14. The system according to claim 13, wherein said individual features of the subject comprises one or more of the following:
personal historical or physical features of the subject;
optometric features of the subject;
biometric features of the subject;
general health features of the subject;
features relative to a visual behavior of the subject,
postural features of the subject.

15. A method for determining a modification or a change of an initial myopia control solution used by a myopic subject, comprising:
determining a real initial value and real ulterior value of an eye state parameter representative of the myopia degree of the eye of the subject at an initial time and at an ulterior time;
providing statistical data relative to the statistical evolution of said eye state parameter with time while said initial myopia control solution is implemented;
determining an expected ulterior value of a magnitude linked to said eye state parameter at said ulterior time based on said statistical data relative to the statistical evolution of said eye state parameter with time while said initial myopia control solution is implemented and a real ulterior value of said magnitude, and a real ulterior value of said magnitude;
comparing said real ulterior value of said magnitude with said expected ulterior value of said magnitude; and
determining said modification or change to said initial myopia control solution for said subject based on the result of this comparison.
